(19) United States
(12) Patent Application Publication
(10) Pub. No.: US 2008/0103154 A1
(43) Pub. Date: May 1, 2008

(54) 3, 4-dihydro-2(IH)-quinolinone and 2(1H)-quinolinone derivatives

(75) Inventor: Roger Tung, Lexington, MA (US)

Correspondence Address: EDWARDS ANGELL PALMER & DODGE LLP, P.O. BOX 55874, BOSTON, MA, 02205, US

(73) Assignee: Concert Pharmaceuticals Inc., Lexington, MA (US)

(21) Appl. No.: 11/895,387

(22) Filed: Aug. 24, 2007

Related U.S. Application Data
(60) Provisional application No. 60/840,269, filed on Aug. 24, 2006.

(51) Int. Cl. A61K 31/497; C07D 401/12; A61P 25/18; A61P 15/00

(52) U.S. Cl. 514/253.07; 544/363

(57) ABSTRACT

The present invention relates to novel 3,4-dihydro-2(1H)-quinolinone and 2(1H)-quinolinone derivatives, their acceptable acid addition salts, solvates, hydrates and polymorphs thereof. The invention also provides compositions comprising a compound of this invention and the use of such compositions in methods of treating diseases and conditions beneficially treated by atypical antipsychotic agents. The invention further provides methods for using a compound of this invention to determine concentrations of a corresponding 3,4-dihydro-2(1H)-quinolinone or 2(1H)-quinolinone compound, particularly in biological fluids, and to determine metabolism patterns of that 3,4-dihydro-2(1H)-quinolinone or 2(1H)-quinolinone compound.

### RELATED APPLICATIONS

This application claims priority to U.S. Provisional Patent Application No. 60/840,269 filed Aug. 24, 2006.

### BACKGROUND OF THE INVENTION

Aripiprazole, chemically described variously as 7-[4-4-(2,3-dichlorophenyl)-1-piperazinylo]butoxy]-3,4-dihydrocarbostyril or 7-[4-4-(2,3-dichlorophenyl)-1-piperazinylo]butoxy]-3,4-dihydro-2(1H)-quinolinone and its pharmaceutically acceptable addition salts, hydrates, and polymorphs thereof, is disclosed as a useful atypical antipsychotic agent. This compound and pharmaceutical compositions comprising it have utility in the treatment of schizophrenia and bipolar mania. Additional disease states that can be treated with aripiprazole include bipolar disorder, acute manic/mixed episode of bipolar disorder, rapid cycling bipolar disorder with or without psychotic symptoms in manic or mixed episodes, major depressive disorder, obsessive compulsive disorder, pediatric conduct disorder, mania, pervasive developmental disorder, autism, Asperger's disorder, alcoholism, agitation, attention deficit/hyperactivity disorder, Alzheimer's dementia, psychosis associated with Alzheimer's dementia, drug-induced psychoses in Parkinson's disease, Tourette's disorder, tic, and drug dependency. See US Food and Drug Administration product label for New Drug Application (NDA) #s 021436, 021713, 021729; Van Peborgh P J et al., Psychopharmacol 2004 18:Abst TC45 & TC46; Riera L et al., Schizophr Res 2004 67: Abst 309; Loze, J Y et al., Eur Neuropsychopharmacol 2004 14; Abst P.2.125; Marder S R et al., J Psychopharmacol 2002 16: Abst A31; Jody D et al., 157^{th} Annu Meet Am Psychiatr Assoc 2004: Abst NR811; Dogin J et al., 158^{th} Annu Meet Am Psychiatr Assoc 2005: Abst NR261; Sanchez R et al., 158^{th} Annu Meet Am Psychiatr Assoc 2005: NR269; Papakostas G1 et al., 158^{th} Annu Meet Am Psychiatr Assoc 2005: NR797; Lippi J R 13^{th} Assoc Eur Psychiatr Congr 2005: Abst O-10-05; Streim J et al., Neurobiol Aging 2005 25: Abst P1-330; Jeste D V et al., J Am Geriatr Soc 2003 51: Abst P543; Radhakrishnan M et al., 158^{th} Annu Meet Am Psychiatr Assoc 2005: Abst NR260; Fernandez H H et al., Clin Neuropharmacol 2004 27: 4; ClinicalTrials.gov Web Site 2005, NCT00198055; ClinicalTrials.gov Web Site 2005, NCT00198107; Beresford T P et al., J Clin Psychopharmacol 2005 25: 363; ClinicalTrials.gov Web Site 2006, NCT002276874; Heimburger G E et al., Mov Disord 2005 20: Abst P561; Hwang T J et al., Eur Neuropsychopharmacol 2005 15; Abst P.3.097; Dillenschneider A et al., Eur Neuropsychopharmacol 2005 15; Abst P.3.122.

Combinations with additional agents have been disclosed to further extend the utility of aripiprazole in the treatment or prevention of: schizophrenia, depression, bipolar depression, depressive disorder, refractive bipolar disorder, autism, alcoholism, cocaine dependency, attention deficit hyperactivity disorder (ADHD), mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity. See: PCT Patent Publications WO2004060374, and WO2006067496, U.S. Pat. No. 6,987,111; and United States patent publication 20050245541.

Aripiprazole has been characterized by in vitro competition binding studies with transfected cell lines expressing rat and human receptors. The compound exhibits high affinity binding to D₂, D₃, 5-HT_{1A} and 5-HT_{2A} along with moderate affinity to D₄, 5-HT_{2C}, 5-HT₇, α1, and H₁ receptors. See Shapiro D A et al., Neuropsychopharmacol. 2003 28: 1400 and Burris K D et al., J Pharmacol Exp Ther 2002 302: 381. Although the affinity of aripiprazole for a number of receptors has been well characterized, the functional effects of aripiprazole are very complex. The actions of aripiprazole vary markedly across receptor systems, such that the activity can be characterized functionally as antagonist, inverse agonist, partial agonist, and full agonist depending upon the receptor system and cell line. This behavior of aripiprazole has been termed “functional selectivity” and proposes that the actions of the compound at a single receptor isoform are highly dependent on the location of the receptor and its signaling partners. See Lawler, C P et al., Neuropsychopharmacol 1999 20:612.

Aripiprazole has also been characterized in a number of animal model systems. For instance, in models of schizophrenia and psychoses (i.e., apomorphine-induced stereotypic behavior in mice and rats and gamma-butyrolactone-induced DOPA synthesis in mice) and models for extra pyramidal syndrome (EPS) side-effects (induction of catalepsy in rodents) have shown results with aripiprazole that are correlated with human clinical effects. See, e.g. Kikuchi T et al., J Pharmacol Exp Ther 1995 274: 329; Oshiro Y et al., J. Med. Chem. 1998 41: 658; and Neuropsychopharmacol 2001 25: 633.

In human clinical studies, Aripiprazole demonstrated good tolerability and statistical efficacy in patients suffering from schizophrenia and bipolar disorder. See US Food and Drug Administration product label for New Drug Application (NDA) #s 021436, 021713, 021729. In comparative trials with risperidone and haloperidol, Aripiprazole showed a better safety and tolerability profile and induced a greater improvement in the negative and positive symptoms of schizophrenia. Aripiprazole has completed or is currently in clinical trials for other indications, such as schizoaffective or schizophreniform disorders, Tourette's disorder, depression, alcoholism, acute agitation associated with schizophrenia, attention deficit/hyperactivity disorder, psychoses associated with Alzheimer's dementia, autism (combination with d-cycloserine), and cocaine dependency. See Van Peborgh, P et al., J Psychopharmacol 2004 18: Abst TC45, TC46; Riera L et al., Schizophr Res 2004 67; Abst. 309;

Following oral administration in humans, Aripiprazole is well absorbed with peak plasma concentrations occurring within 3 to 5 hours; the absolute oral bioavailability of the oral formulation is 87%. Aripiprazole is extensively metabolized in humans. One metabolite, 7-[4-4-(2,3-dichlorophenyl)-1-piperazinylo]butoxy]-2(1H)-quinolinone (“dehydroaripiprazole”), derived by dehydrogenation of the quinolinone ring is equipotent with aripiprazole and accounted for 20% of activity. See US FDA approved label for NDA#021436, approved Feb. 16, 2006.

Despite the beneficial activities of aripiprazole, there is a continuing need for new compounds to treat the aforementioned diseases and conditions.

### DETAILED DESCRIPTION OF THE INVENTION

The terms “ameliorate” and “treat” are used interchangeably and both mean decrease, suppress, attenuate, diminish, arrest, or stabilize the development or progression of a disease (e.g., a psychotic disorder).

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of aripiprazole or dehydroaripiprazole will inherently contain small amounts of deuterated and/or ¹³C-containing isotopologues. The concentration of naturally abundant stable hydrogen and carbon isotopes, notwithstanding this variation, is small and immaterial as compared to the degree of stable isotopic substitution of compounds of this invention. See for instance Wada E et al., Seikagaku 1994, 66:15; Ganes L Z et al., Comp Biochem Physiol Mol Integr Physiol 1998, 119:725. The compounds of the present invention are distinguished from such naturally occurring minor forms in that the term “compound” as used in this invention refers to a composition of matter that is predominantly a specific isotopologue.

In one embodiment, a “compound”, as defined herein, contains less than 10%, preferably less than 6%, and more preferably less than 3% of all other isotopologues. These limits of isotopic composition, and all references to isotopic composition herein, refer solely to the relative amounts of deuterium/hydrogen and ¹³C/¹²C present in the active, free base form of the compound of Formula I or II, and do not include the isotopic composition of counterions.

In other embodiments, when a particular position is designated as having deuterium in a compound of this invention, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In still other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to species that differ from a specific compound of this invention only in the isotopic composition of their molecules or ions.

The term “compound” as used herein, is also intended to include salts, solvates, and hydrates of that compound. The specific recitation of “salt,” “solvate,” or “hydrate,” in certain aspects of the invention described in this application shall not be interpreted as an intended omission of these forms in other aspects of the invention where the term “compound” is used without recitation of these other forms.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention contain one or more asymmetric carbon atoms. As such, a compound of this invention can exist as the individual stereoisomers (enantiomers or diastereomers) as well a mixture of stereoisomers. Accordingly, a compound of the present invention will include not only a stereoisomeric mixture, but also individual respective stereoisomers substantially free from one another stereoisomers. The term “substantially free of other stereoisomers” as used herein means less than 25% of other stereoisomers, preferably less than 10% of other stereoisomers, more preferably less than 5% of other stereoisomers and most preferably less than 2% of other stereoisomers, are present. Methods of obtaining or synthesizing diastereomers are well known in the art and may be applied as practicable to final compounds or to starting material or intermediates. Other embodiments are those wherein the compound is an isolated compound.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to atypical antipsychotic agents).

The term “heavy atom” refers to isotopes of higher atomic weight than the predominant naturally occurring isotope.

By “reference” is meant a standard or control condition.

Both “²H” and “D” refer to deuterium.

“Stereoisomer” refers to both enantiomers and diastereomers.

“tert” refers to tertiary.

“US” refers to the United States of America.

“FDA” refers to Food and Drug Administration.

“NDA” refers to New Drug Application.

“CYP3A4” refers to cytochrome P450 oxidase isoform 3A4.

“CYP2D6” refers to cytochrome P450 oxidase isoform 2D6.

“5-HT” refers to 5-hydroxytryptamine or serotonin.

Throughout this specification, reference to “each Y” includes, independently, all “Y” groups (Y¹, and Y²), and reference to “each Z” includes, independently, all “Z” groups (Z¹, Z², Z³, and Z⁴) where applicable.

Therapeutic Compounds

The present invention provides a compound of formula I: 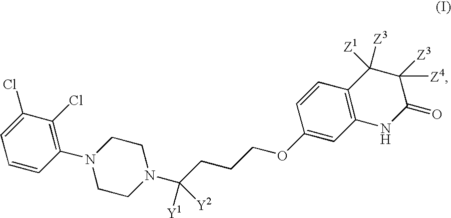
or a salt, hydrate, or solvate thereof,
wherein:

each Y is independently selected from hydrogen, deuterium, and fluorine;

each Z is independently selected from hydrogen, deuterium, and fluorine; and

at least one Y or Z is deuterium.

In one embodiment Y¹ and Y² are the same.

In another embodiment Z¹ and Z² are the same.

In yet another embodiment, Z³ and Z⁴ are the same.

In a more specific embodiment, Y¹ and Y² are simultaneously deuterium.

In another more specific embodiment Z¹ and Z² are simultaneously deuterium.

In yet another more specific embodiment, Z³ and Z⁴ are simultaneously deuterium.

In a still more specific embodiment, Y¹, Y², Z¹ and Z² are simultaneously deuterium.

In another even most specific embodiment, Y¹ and Y² are simultaneously deuterium; and Z¹ and Z² are simultaneously fluorine.

In certain embodiments, the compound of formula I comprises two or more deuterium atoms.

In other embodiments, the compound of formula I comprises three or more deuterium atoms.

In yet other embodiments, the compound of formula I comprises two or more deuterium atoms and at least one fluorine atom.

In other embodiments, the compound of formula I, comprises two or more deuterium atoms and two or more fluorine atoms.

The present invention also provides a compound of formula II: 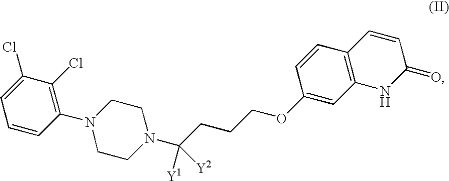
or a salt, hydrate, or solvate thereof;
wherein:

one Y is deuterium; and

the other Y is independently selected from hydrogen, deuterium, and fluorine.

In one embodiment, Y³ and Y⁴ are simultaneously deuterium. In another embodiment, one Y is deuterium and the other Y is fluorine.

In a specific embodiment, the compound of this invention is selected from: 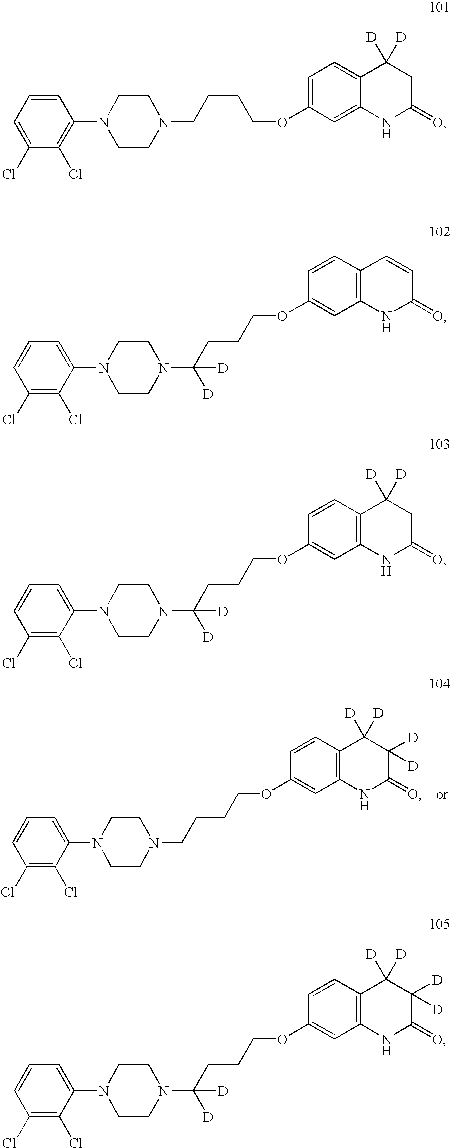
and an HCl salt of any of the foregoing.

In another set of embodiments, in each embodiment set forth above, any atom not designated as deuterium is present at its natural isotopic abundance.

In still another embodiment, a salt of formula I or II is an HCl salt.

The compounds of the invention may be synthesized by well-known techniques. The starting materials and certain intermediates used in the synthesis of the compounds of this invention are available from commercial sources or may themselves be synthesized using reagents and techniques know in the art. For instance, routes to the all hydrogen isotopologues of this invention are described in U.S. Pat. Nos. 5,006,528, and 6,995,264; PCT Patent Publication Nos. WO2006038220 and WO2005077904; and in Oshiro Y et al., J Med Chem, 1998, 41:658. Each of the publications, patents, and patent applications referred to herein is incorporated in its entirety by reference.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

Methods of incorporating deuterium in target compounds are extensively documented. See, for instance, The Journal of Labelled Compounds and Radiopharmaceuticals (John Wiley & Sons), most issues of which contain detailed experimental descriptions on specific incorporation of deuterium into bio active small organic molecules. See also, for instance, Leis H J, Curr Org Chem, 1998, 2:131 and reference therein, and Moebius G, ZfI-Mitteilungen 1989 150:297. Suitable commercial supplies of deuterium-labeled reagents include, among others, Isotec, Inc. (Miamisburg, Ohio); Cambridge Isotope Laboratories (Andover, Mass.); ICON Services Inc. (Summit, N.J.); and C/D/N Isotopes, Inc. (Pointe-Claire, Quebec, Canada).

Exemplary Synthesis

A convenient method for synthesizing compounds of Formula I is depicted in Scheme 1. 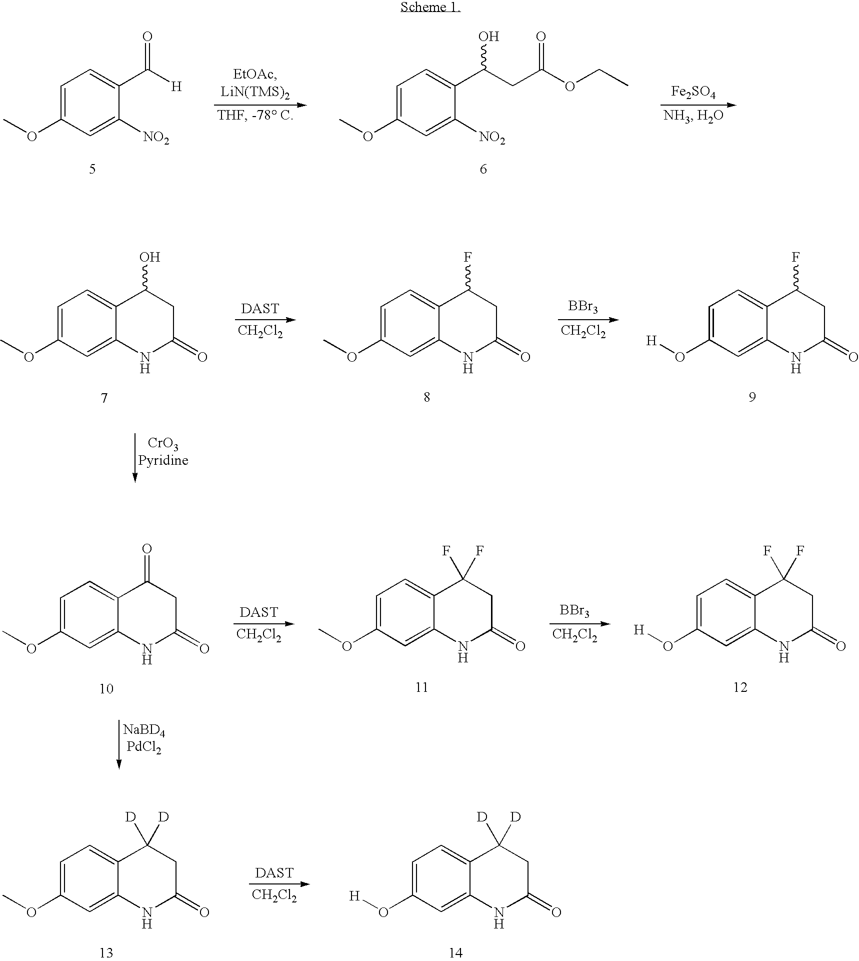
Relevant deuterium and fluorine substituted starting materials may also be readily synthesized. For instance, as shown in Scheme 1, 2-nitro-4-methoxy benzaldehyde (5) can be reacted with the enolate of ethyl acetate to yield 6. Reduction of the nitro 6 with ferrous sulfate and subsequent cyclization provides the 4-hydroxy quinolinone 7, which can then be readily converted to the 4-fluoro derivative 8 using a fluorination agent such as DAST. Compound 7 can also be oxidized to ketone 10 with chromium trioxide/pyridine, which can then be converted using DAST to the difluoro derivative 11. Alternatively, the ketone 10 can be reduced with sodium borodeuteride in the presence of PdCl2 to provide the dideutero compound 13. Compounds 8, 11, and 13 are then readily reacted with boron tribromide to provide the phenols 9, 12, and 14, respectively.

Additional deuterium substituted starting materials may also be readily synthesized. For instance, as shown in Scheme 2.

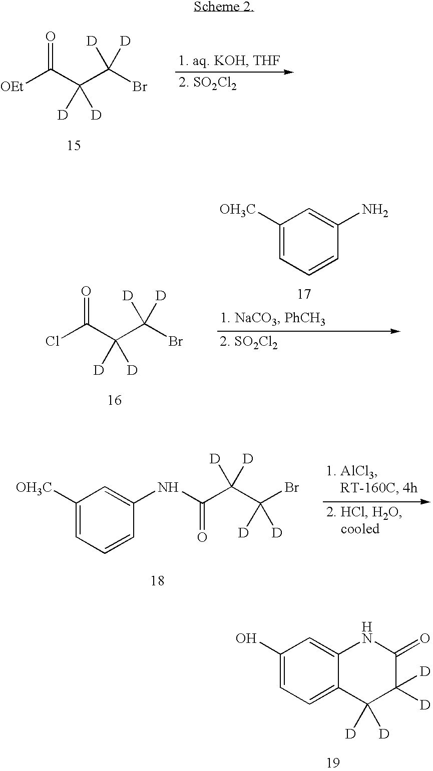

Commercially available deuterated ethyl-2-bromo-propionate (15) is converted to the acid chloride 16, which is then reacted with 3-methoxyaniline 17 to provide the amide 18. Cyclization under Friedel-Crafts conditions provides the desired deuterated carbostyryl derivative 19.

Additional appropriately deuterated and fluorinated starting materials can be prepared as shown in Scheme 3.

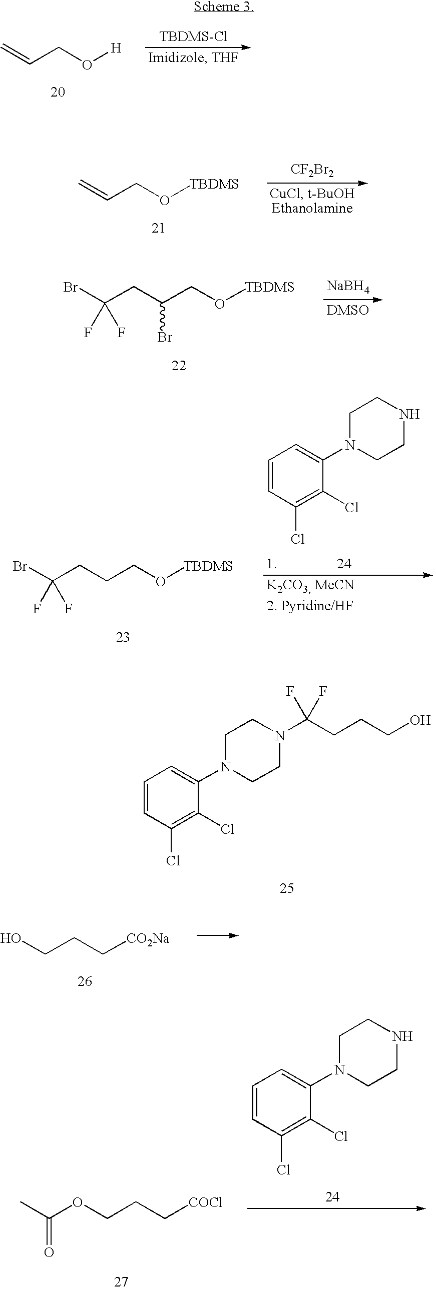

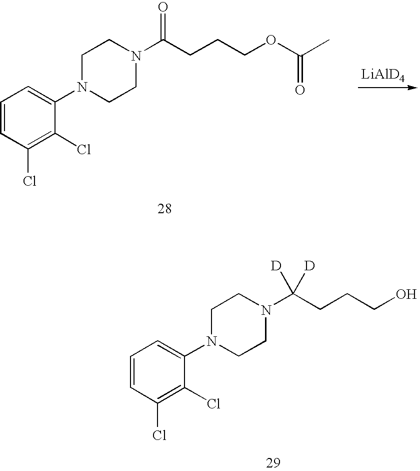

Commercially available allyl alcohol (20) is converted to the silyl ether 21 prior to reaction with CF₂Br₂ in the presence of cuprous chloride to provide 22. See Burton, D J et al., J Org Chem 1970, 35:1339. Selective reduction of the secondary carbon-bromine bond to yield 23 can be effected using sodium borohydride in dimethylsulfoxide under mild conditions. See Gonzalez, J et al., J Org Chem 1991, 56:4322. N-alkylation followed by cleavage of the silyl ether provides 25. The deuterated version of compound 25 is obtained from commercially available sodium hydroxybutyrate, which is converted to acetate protected acid chloride 27 by known procedures. See Nishi, T et al., Chem Pharm Bull, 1985, 33:1140. Compound 27 is then reacted with 24 to provide the amide 28, which can then be simultaneously deprotected and reduced to the di-deutero amine 29 with lithium aluminum deuteride.

The final compounds of this invention can be prepared according to Schemes 4 or 5.

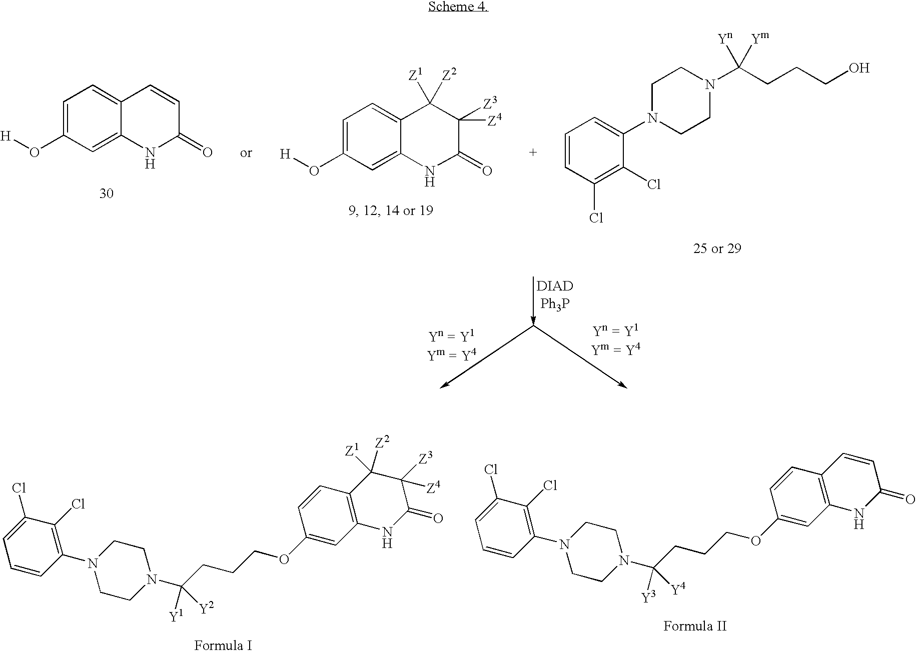

Compounds 25 or 29 are coupled using the Mitsunobu reaction on phenols 9, 12, 14, or 19 to provide compounds of Formulae I or II.

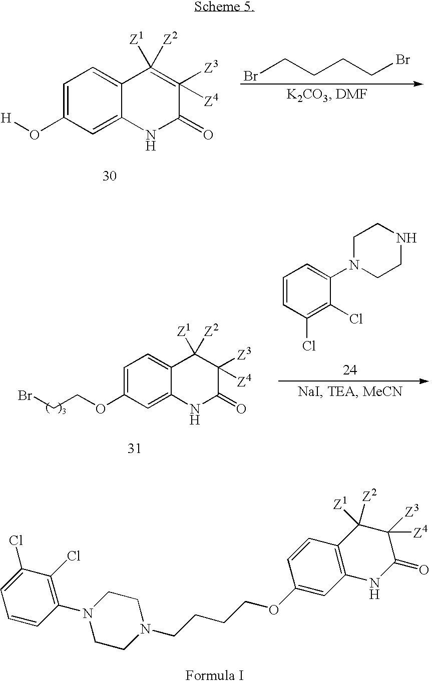

In another approach, compounds of generic formula 30 (e.g., compounds 9, 12, 14 or 19) can be reacted with 1,4-dibromobutane to provide the 4-bromobutylethers of formula 31. These alkyl bromides 31 can then be reacted with amine 24 to provide final compounds of Formula I. See Oshiro, Y et al., J Med Chem, 1998, 41:658.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Additional methods of synthesizing compounds of formulae I and II and their synthetic precursors, including those within routes not explicitly shown in schemes herein, are within the means of chemists of ordinary skill in the art. Methods for optimizing reaction conditions, if necessary minimizing competing by-products, are known in the art. Reaction optimization and scale-up may advantageously utilize high-speed parallel synthesis equipment and computer-controlled microreactors (e.g. Design and Optimization in Organic Synthesis, 2nd Edition, Carlson R, Ed, 2005; Elsevier Science Ltd.; Jahnisch, K et al, Angew. Chem. Int. Ed. Engl. 2004 43: 406; and references therein). In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database.

The synthetic methods described herein may also additionally include steps, either before or after any of the steps described in the preceding schemes, to add or remove suitable protecting groups in order to ultimately allow synthesis of the compounds described herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

Compositions

The invention also provides compositions comprising an effective amount of a compound of Formulae I or II, or a pharmaceutically acceptable salt, solvate, or hydrate, thereof; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with an atypical antipsychotic agent. Preferably, the second therapeutic agent is an agent known to treat one or more of schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, or frigidity.

The term “psychotic disorder” includes schizophreniform diseases, schizoaffective disorders, delusional disorders, effective disorders, tic disorders, depression with psychotic features, chronic schizophrenic psychoses, schizoaffective psychoses, and temporary acute psychotic disorders. The term “schizophrenia” as used herein refers to a number of disease states, such as schizoaffective disorder, schizophreniform disorder, paranoid type schizophrenia, disorganized type schizophrenia, catatonic type schizophrenia, undifferentiated type schizophrenia, prodromal schizophrenia, and residual type schizophrenia.

In one embodiment, the second therapeutic agent is selected from an NK3 receptor antagonist; a Gly Transporter Type I inhibitor, such as disclosed in International Patent Publication WO2006000222 and WO2006066121; memantine; an AMPA receptor potentiator, such as disclosed in International Patent Publication WO2005013961; a GABA modulator, anticonvulsant, or benzodiazepine; an antidepressant; a nicotinic receptor agonist or antagonist; a serotonin reuptake inhibitor; sabcomeline, an M1/M4 receptor agonist; an opioid antagonist; D-cycloserine; Lamotrigine; methylphenidate; divalproex; clozapine; H1-receptor agonist such as disclosed in US Patent Publication 20060148787; an adenosine A2a receptor antagonist such as disclosed in US Patent Publication 20060128694; COX-2 inhibitor; an azabicyclo compound such as described in US Patent Publication 20050171086; flibanserin; lithium, or a pharmaceutically acceptable salt, solvate, hydrate, polymorph, or prodrug of any of the said second therapeutic agents and combinations of the foregoing.

Examples of NK3 receptor antagonists include, but are not limited to, talnetant and osanetant.

Examples of GABA modulators, anticonvulsants, and benzodiazepines include, but are not limited to, alprazolam, baclofen, bentazepam, bretazenil, bromazepam, brotizolam, brotizolam, camazepam, carbamazepine, chlorazepate, chlordiazepoxide, chlorodiazepam, cinolazepam, clobazam, clonazepam, clotiazepam, cloxazolam, clozapin, delorazepam, diazepam, dibenzepin, dipotassium chlorazepam, divaplon, estazolam, ethosuximide, ethyl-loflazepate, etizolam, felbamate, fludiazepam, flumazenil, flunitrazepam, flurazepam-HCl, flutoprazepam, gabapentin, halazepam, haloxazolam, imidazenil, ketazolam, lamotrigine, levetiracetam, loprazolam, lorazepam, lormetazepam, medazepam, metaclazepam, methexamide, mexazolam, midazolam-HCl, muscimol, nabazenil, nimetazepam, nitrazepam, nordazepam, oxazepam-tazepam, oxazolam, oxcarbazepine, phenyloin, pinazepam, prazepam, pregabalin, progabide, quazepam, riluzole, sarmazenil, suriclone, temazepam, tetrazepam, tiagabine, tofisopam, triazolam, valproate, vigabatrin, zaleplon, zolazepam, zolpidem, zonisamide, and zopiclone.

Examples of antidepressants include, but are not limited to, fluoxetine, paroxetine, norfluoxetine, sertraline, fluvoxamine, citalopram, escitalopram, bupropion, nefazodone, mirtazapine, venlafaxine, duloxetine, milnacipran, reboxetine, zimelidine, indalpine, gepirone, femoxetine, and alaproclate.

Examples of nicotinic receptor antagonists include, but are not limited to, amantadine, hexamethonium, erysodine, pempidine, methyllycaconitine, chlorisondamine, trimethaphan, mecamylamine, dimecamine, erysodine, 18-methoxycoronaridine, bupropion, dextromethorphan, dextrorphan and ibogaine.

Examples of nicotinic receptor agonists include, but are not limited to, varenicline and nicotine derivatives as disclosed in U.S. Pat. Nos. 5,242,934; 5,223,497; 5,278,045; 5,232,933; 4,965,074; 5,278,176; and 5,227,391.

Examples of serotonin reuptake inhibitors include, but are not limited to, fluoxetine, duloxetine, venlafaxine, milnacipran, citalopram, fluvoxamine, paroxetine, sertraline, and escitalopram.

Examples of opioid receptor antagonists include, but are not limited to, naltrexone, naloxone, and nalmefene.

Examples of COX-2 inhibitors include, but are not limited to, celecoxib, rofecoxib, meloxicam, piroxicam, deracoxib, parecoxib, valdecoxib, etoricoxib, COX189, ABT963, and JTE-522.

Preferably the second therapeutic agent co-formulated with a compound of this invention is an agent useful in the treatment of schizophrenia, bipolar disorder, ADHD, or autism. More preferably, the agent is selected from clozapine, depakote ER, lamotrigine, methylphenidate, and D-cycloserine.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of a disorder effectively treated by an atypical antipsychotic agent, prevent the advancement of a disorder effectively treated by an atypical antipsychotic agent, cause the regression of a disorder effectively treated by an atypical antipsychotic agent, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537. An effective amount of a compound of this invention can range from about 0.001 mg/kg to about 500 mg/kg, more preferably 0.01 mg/kg to about 50 mg/kg, more preferably 0.1 mg/kg to about 2.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents listed above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

Methods of Treatment

Another embodiment of the invention is a method of treating a subject suffering from or susceptible to a disease that is beneficially treated by an atypical antipsychotic agent comprising the step of administering to said subject an effective amount of a compound or a composition of this invention. Such diseases include but are not limited to, schizophrenia, bipolar disorder (including acute manic/mixed episode of bipolar disorder and rapid cycling bipolar disorder with or without psychotic symptoms in manic or mixed episodes), bipolar mania, autism, alcoholism, agitation, attention deficit/hyperactivity disorder, anxiety, behavioral disorder, dementia, Alzheimer's dementia, Asperger's disorder, conduct disorder, depression, drug dependency, insulin resistance, mania, obsessive-compulsive disorder, Parkinson's disease, psychosis associated with dementia, drug-induced psychosis, pervasive developmental disorder, prodromal schizophrenia, prodromal psychoses, schizoaffective disorder, major depressive disorder, social anxiety, tic, and Tourette's disorder. Other embodiments include any of the methods herein wherein the subject is identified as in need of the indicated treatment.

In a preferred embodiment, the method of this invention is used to treat a subject suffering from or susceptible to a disease or condition selected from schizoaffective or schizophreniform disorders, Tourette's disorder, depression, alcoholism, acute agitation associated with schizophrenia, attention deficit/hyperactivity disorder, psychoses associated with Alzheimer's dementia, and cocaine dependency. More preferably the method is used to treat a patient suffering from schizophrenia, mania and bipolar disorder.

In another embodiment, the above method of treatment comprises the further step of co-administering to said patient one or more second therapeutic agents which, alone or in combination with aripiprazole, are effective to treat one or more of: schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity.

Such second therapeutic agents useful for co-administration with the compounds of Formula I or II are described above. In a specific embodiment, the second therapeutic agent is selected from clozapine, depakote ER, lamotrigine, methylphenidate, and D-cycloserine.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of formula I or II alone or together with one or more of the above-described second therapeutic agents in the manufacture of a medicament, either as a single composition or as separate dosage forms, for treatment or prevention in a subject of a disease, disorder or symptom set forth above.

Diagnostic Methods and Kits

According to another embodiment, the invention provides a method of determining the concentration of aripiprazole in a biological sample, said method comprising the steps of:

a) adding a known concentration of a compound of Formula I to the biological sample;

b) subjecting said biological sample to a measuring device that distinguishes the aripiprazole from the compound of Formula I;

c) calibrating the measuring device to correlate the detected quantity of aripiprazole with the known concentration of the compound of Formula I added to said biological sample;

d) measuring the quantity of aripiprazole in the biological sample with said calibrated measuring device; and

e) determining the concentration of aripiprazole in the solution of sample using the correlation between detected quantity and concentration obtained for a compound of Formula I.

A similar method can be used to determine the concentration of dehydroaripiprazole in a biological sample by simply substituting a compound of Formula II for the compound of Formula I.

Measuring devices that can distinguish aripiprazole or dehydroaripiprazole from a compound of Formula I or II, respectively include any measuring device that can distinguish between two compounds that are of identical structure except that one contains one or more heavy atom isotope versus the other. Preferably, such a measuring device is a mass spectrometer, NMR spectrometer, or IR spectrometer.

In another embodiment, the invention provides a method of evaluating the metabolic stability of a compound of formula I or II, comprising the steps of contacting the compound of formula I or II, or its acid addition salt with a metabolizing enzyme source for a period of time; and comparing the amount of said compound and metabolic products of said compounds after said period of time.

In a related embodiment, the invention provides a method of evaluating the metabolic stability of a compound of Formulae I or II in a patient following administration of the compound. This method comprises the steps of obtaining a serum, urine or feces sample from the patient at a period of time following the administration of the compound of Formula I or II to the subject; and comparing the amount of the compound of Formula I or II with the metabolic products of the compound of Formula I or II, respectively, in the serum, urine or feces sample.

The present invention also provides kits for use in treating schizophrenia, mania, or bipolar disorder. These kits comprise (a) a pharmaceutical composition comprising a compound of Formula I, or a compound of Formula II, wherein said pharmaceutical composition is in a container; and (b) instructions describing a method of using the pharmaceutical composition to treat schizophrenia, mania, or bipolar disorder.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Synthesis of 7-[4-[4-(2,3-Dichlorophenyl)-1-piperazinyl]butoxy]-4-d₂-3,4-dihydro-2(1H)-quinolinone (Compound 101)

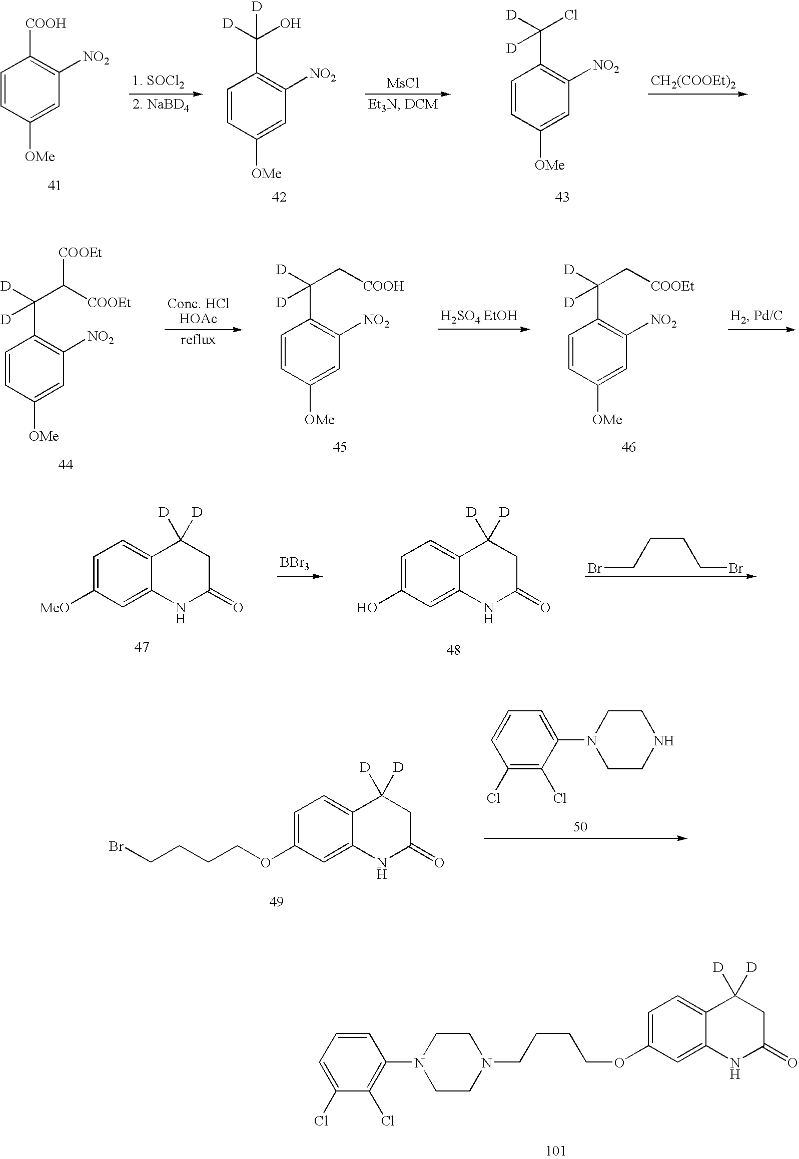

Compound 42: To a solution of compound 41 (177.41 g, 0.90 mol) in dry toluene (1.5 L) was added SOCl₂ (197.0 mL, 2.70 mol) at room temperature (“RT”). The resulting mixture was heated under reflux conditions for 3 h under an atmosphere of nitrogen and the solvent was then evaporated in vacuo. To a suspension of NaBD₄ (37.8 g, 0.90 mol) in 1:1 THF/DMF (IL) was added a solution of the acid chloride in THF (500 mL) at 0° C., and the resulting mixture was stirred at RT overnight. The reaction was quenched by the addition of 10% HCl solution, and then extracted with EtOAc. The combined organic layers were washed twice with saturated NaHCO₃ solution, then with brine, dried over anhydrous Na₂SO₄, filtered, and then concentrated in vacuo. The residue was triturated with 10% EtOAc/heptane to afford compound 2 (171.8 g) as a yellow solid.

Compound 43: To a solution of compound 42 (171.8 g, ˜0.90 mol) in DCM (3 L) was added Et₃N (163 mL, 1.17 mol) and Ms—Cl (76.6 mL, 0.99 mol) at 0° C. The resulting mixture was stirred at RT overnight. The reaction was quenched by the addition of water, washed with brine, dried over anhydrous Na₂SO₄, filtered, and concentrated in vacuo. The residue was subjected to flash chromatography (2.5 kg silica gel, eluted by 10% to 15% EtOAc/heptane in gradient) to afford compound 3 (116.8 g, 64% yield over two steps) as a yellow oil.

Compound 44: To a suspension of NaH (60% dispersion in mineral oil, 26.52 g, 0.6629 mmol) in 1:1 DMF/THF (400 mL) was added a solution of diethyl malonate (127.0 mL, 0.8363 mol) in THF (500 mL) at −10° C. The mixture was stirred for 30 min at 0° C. To this mixture was added a solution of compound 43 (112.5 g, 0.5524 mol) in THF (500 mL) dropwise over 2.5 h at −10° C., followed by stirring at RT overnight. The reaction was quenched by the addition of 2M HCl solution, and the layers were separated. The aqueous layer was extracted with EtOAc. The combined organic layers were washed with brine, dried over anhydrous Na₂SO₄, filtered, and concentrated in vacuo. The residue was subjected to flash chromatography (Biotage-150, eluted by 10% EtOAc/heptane) to afford the title compound 4 (131.0 g, 70% yield) as a white solid.

Compound 45: A mixture of compound 44 (129.0 g, 0.3941 mol) in acetic acid (700 mL) and conc. HCl solution (200 mL) was heated under reflux conditions for 8 h. The reaction mixture was concentrated in vacuo to give the crude compound 45 as an off-white solid.

Compound 46: A mixture of compound 45 (˜0.394 mol) and conc. H₂SO₄ (4 mL) in EtOH (1.8 L) was heated under reflux conditions for 4 h. The reaction mixture was concentrated in vacuo to give the crude compound 46 as a yellow oil.

Compound 47: A mixture of compound 46 (˜0.394 mol) and 10% Pd/C (50% wet, 15 g) in acetic acid (1.15 L) was subjected to hydrogenation @ 20 psi at RT for 1 h. The mixture was filtered through celite, then washed with ethanol. The filtrate was heated under reflux conditions for 1 h to drive the cyclization reaction to completion. The mixture was concentrated in vacuo, the residue was diluted with EtOAc, then washed with saturated NaHCO₃ solution. The aqueous phase was extracted with EtOAc and the combined organics were washed with brine, dried over anhydrous Na₂SO₄, filtered, and concentrated in vacuo to afford compound 47 (67.0 g, 93% yield over 3 steps) as a brown solid.

Compound 48: To a solution of compound 47 (15.23 g, 85 mmol) in DCM (425 mL) was added BBr₃ (1.0M solution in THF, 425 mL) at −70° C. The resulting mixture was allowed to warm to RT then was stirred overnight. The reaction was quenched by the addition of water at 0° C., and the precipitate was filtered, washed with water, then dried under vacuum at 60° C. to afford compound 48 (12.2 g, 87% yield) as an off-white solid.

Compound 49: To a solution of compound 48 (3.3 g, 20 mmol) in isopropanol (70 mL) was added KOH (1.4 g, 25 mmol) at RT, followed by 1,4-dibromobutane (7.2 mL, 60 mmol). The reaction mixture was heated under reflux conditions overnight. The resulting precipitate was filtered off, and the filtrate was concentrated in vacuo. The residue was triturated with 1:1 EtOAc/heptane to give compound 49 (3.8 g, 63% yield) as an off-white solid.

Compound 101: To a mixture of compound 49 (6.0 g, 20 mmol) and 1-(2,3-dichloro-phenyl)-1-piperazine monohydrochloride (50; 5.35 g, 20 mmol) in acetonitrile (150 mL) was added Et₃N (7 mL, 50 mmol) at RT. The resulting mixture was heated under reflux conditions for 1 h, then was concentrated in vacuo. The residue was subjected to flash chromatography (silica gel with dry-loading, eluted by 50% EtOAc/heptane to EtOAc to 10% MeOH/EtOAc in gradient) to afford compound 101 as an off-white solid. ¹H-NMR (300 MHz, DMSO-d₆): δ 1.74-1.89 (m, 4H), 2.4 (s, 2H), 3.17-3.23 (m, 6H), 3.42-3.45 (m, 2H), 3.58-3.60 (m, 2H), 3.94 (t, J=5.8 Hz, 2H), 6.45 (d, J=2.6 Hz, 1H), 6.50 (dd, J₁=8.2 Hz, J₂=2.3 Hz, 1H), 7.06 (d, J=8.2 Hz, 1H), 7.19-7.23 (m, 1H), 7.36-7.38 (m, 2H), 9.99 (s, 1H), 10.66 (bs, 1H). ¹³C-NMR (75 MHz, DMSO-d₆): δ 20.86, 26.60, 31.25, 48.41, 51.81, 55.92, 67.42, 102.47, 108.20, 116.27, 120.52, 125.99, 126.76, 129.08, 129.34, 133.43, 139.96, 150.20, 158.40, 170.97. HPLC (method: 20 mm C18-RP column; mobile phase: 2-95% ACN+0.1% formic acid in 3.3 min with 1.7 min hold at 95% ACN, Wavelength: 254 nm): retention time: 2.83 min. MS (M+H⁺): 450.2. Elemental Analysis (C₂₃H₂₅D₂Cl₂N₃O₂.1HCl.1.75H₂O): Calculated: C=53.29, H=6.12, N=8.11. Found: C=53.59, H=6.08, N=7.91.

### Synthesis of 7-[4-[4-(2,3-Dichlorophenyl)-1-piperazinylo]-4-d₂-butoxy]-2(1H)-quinolinone hydrochloride salt (Compound 102 HCl Salt)

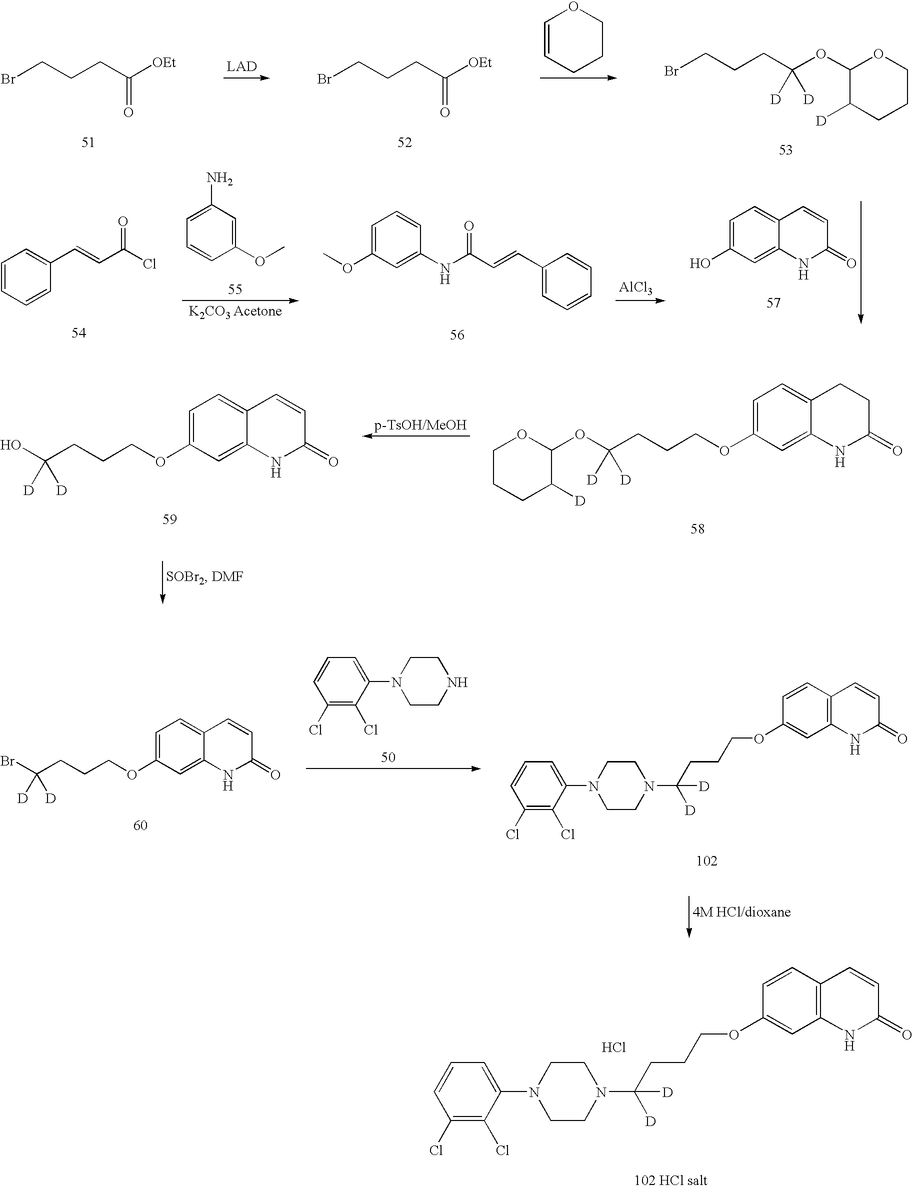

Compound 52: To a mixture of LiAlD₄ (30.4 g, 0.724 mol) in anhydrous ether (1.5 L) was added ethyl 4-bromobutyrate (51; 141.2 g, 0.724 mol) at −10 to 0° C., and the resulting mixture was stirred at 0° C. for 1 h. The reaction was quenched with D₂O (30 mL), 15% NaOD solution in D₂O (30 mL) and D₂O (90 mL) under the cooling of an ice-bath. The white solid was removed by filtration, and the resulting filtrate was dried over anhydrous Na₂SO₄, filtered, and concentrated in vacuo to give compound 52 (82 g) as a pale-yellow oil. ¹H NMR showed that this crude product was a mixture (3:1 ratio) of compound 52 and d₃-butanol.

Compound 53: To a solution of crude compound 52 (66.3 g, 0.425 mol) in ether was added p-TsOH.H₂O (0.15 g) and 3,4-dihydro-2H-pyran (50 mL, 0.548 mol) at 0° C., and the resulting mixture was stirred at RT for 2 h. The mixture was washed with saturated NaHCO₃ solution, brine, dried over anhydrous Na₂SO₄, filtered, and concentrated in vacuo to provide compound 53 (90.3 g) as a pale-yellow oil.

Compound 56: To a stirred solution of m-anisidine (55; 24.6 g, 0.20 mol), K₂CO₃ (41.5 g, 0.30 mol), water (400 mL) and acetone (200 mL) was added cinnamoyl chloride (54; 41.0 g, 0.25 mol) at 0° C. The resulting mixture was stirred at 0° C. for 0.5 h, and then poured into ice-water (1 L). The mixture was extracted with EtOAc, and the combined organics were washed with brine, dried over anhydrous Na₂SO₄, filtered, and concentrated in vacuo to afford compound 56 (53.2 g) as a pale-yellow solid.

Compound 57: To a suspension of compound 56 (45.54 g, 0.18 mol) in chlorobenzene (1.35 L) at 0° C. was added AlCl₃ (144.2 g, 1.08 mol) portionwise. The resulting mixture was gradually warmed to 120° C., was stirred at 120° C. for 2 h and then cooled to RT. The mixture was poured into ice-water (4.5 L) and the resulting precipitate was removed by filtration then dried under vacuum at 50° C. to afford compound 57 (23.5 g, 81% overall yield for 2 steps) as an off-white solid.

Compound 58: To a mixture of compound 57 (6.45 g, 40 mmol) in DMF (150 mL) was added NaOH (1.76 g, 44 mmol) and bromide 53 (28.8 g, 120 mmol) at RT. The resulting mixture was stirred at 50-60° C. for one day, then was diluted with EtOAc and washed by water. The aqueous layer was then extracted with EtOAc and the combined organics were washed with brine, dried over anhydrous Na₂SO₄, filtered, and concentrated in vacuo to give the crude compound 58.

Compound 59: To a mixture of crude compound 58 (˜40 mmol) in methanol (500 mL) was added p-TsOH.H₂O (1.5 g, 4 mmol) at RT. The resulting mixture was stirred at RT for 6 h, then was concentrated in vacuo to remove most of the methanol. The residue was diluted with EtOAc, washed with saturated NaHCO₃ solution, and the resulting aqueous phase was extracted with EtOAc. The combined organics were washed with brine, dried over anhydrous Na₂SO₄, filtered, and concentrated in vacuo to provide compound 58 (5.0 g, 53% yield over 2 steps) as a reddish brown solid. (GB-12-50)

Compound 60: To a mixture of compound 59 (4.5 g, 19.126 mmol) in DMF (75 mL) was added SOBr₂ (3.0 mL, 38.252 mmol) at 0° C. The reaction mixture was stirred at 0° C. for 2 h then was diluted with water. The resulting solid was filtered, washed with water, and dried under vacuum at 50° C. to give compound 60 (3.65 g) as a reddish-brown solid.

Compound 102: A mixture of compound 60 (3.65 g, 12.248 mmol) and NaI (2.75 g, 18.372 mmol) in acetonitrile (300 mL) was heated under reflux conditions for 0.5 h, then was cooled to RT. To this mixture was added 1-(2,3-dichlorophenyl)piperazine hydrochloride (3.25 g, 13.473 mmol) and Et₃N (1.9 mL, 13.473 mmol) at RT, and the resulting mixture was heated under reflux conditions for 3 h. The mixture was allowed to cool to RT, the precipitate was removed by filtration, and the filtrate was adsorbed on silica gel (50 g) and concentrated in vacuo. The residue was subjected to flash chromatography (150 g silica gel with dry-loading, eluted by EtOAc to 5% MeOH/EtOAc in gradient) to afford the desired compound 102 as an off-white solid. Purification was repeated to ensure optimal purity prior to the next step

Compound 102 HCl salt: A mixture of compound 101 (1.0 g) in 4M HCl/dioxane (100 mL) was stirred at RT for 2 h. Solvent was removed in vacuo and the remaining residue was dried under vacuum at 50° C. to afford the target compound 101 HCl salt (1.0 g) as an off-white solid. ¹H-NMR (300 MHz, DMSO-d₆): δ 1.82-1.90 (m, 4H), 3.12-3.25 (m, 4H), 3.45 (d, J=9.1 Hz, 2H), 3.61 (d, J=8.5 Hz, 2H), 4.06 (t, J=5.4 Hz, 2H), 6.31 (d, J=9.6 Hz, 1H), 6.81-6.83 (m, 2H), 7.22 (dd, J₁=7.0 Hz, J₂=2.6 Hz, 1H), 7.34-7.41 (m, 2H), 7.58 (d, J=9.4 Hz, 1H), 7.83 (d, J=9.6 Hz, 1H), 10.49 (bs, 1H), 11.68 (s, 1H). ¹³C-NMR (75 MHz, DMSO-d₆): δ 20.64, 26.41, 48.44, 51.74, 67.02, 67.65, 99.32, 111.46, 114.08, 119.23, 120.53, 126.02, 126.72, 129.37, 129.99, 133.42, 140.75, 141.30, 150.19, 160.95, 162.93. HPLC (method: 20 mm C18-RP column; mobile phase: 2-95% ACN+0.1% formic acid in 3.3 min with 1.7 min hold at 95% ACN, Wavelength: 254 nm): retention time: 2.75 min. MS (M+H⁺): 448.2. Elemental Analysis (C₂₃H₂₃D₂Cl₂N₃O₂.HCl.3H₂O): Calculated: C=51.07, H=6.34, N=7.77. Found: C=50.69, H=5.84, N=7.50.

### Synthesis of 7-[4-[4-(2,3-Dichlorophenyl)-1-piperazinylo]-4-d₂-butoxy]-4-d₂-3,4-dihydro-2(1H)-quinolinone hydrochloride salt (Compound 103 HCl Salt)

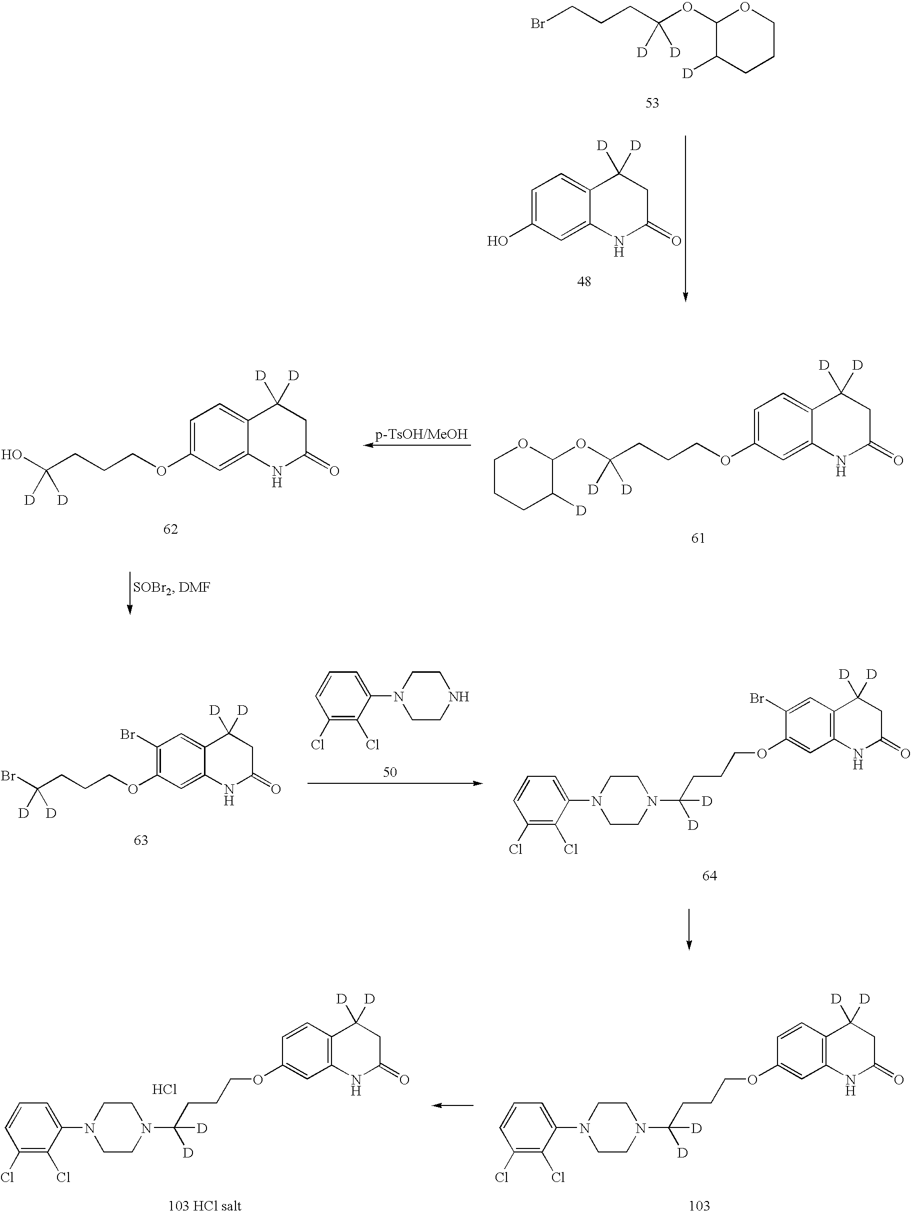

Compound 61: To a mixture of compound 48 (6.60 g, 40 mmol) in DMF (150 mL) was added NaOH (1.76 g, 44 mmol) and bromide 53 (28.8 g, 120 mmol) at RT. The reaction mixture was stirred at 50-60° C. for one day and the resulting mixture was concentrated in vacuo to remove most of the DMF. The residue was diluted with EtOAc, washed with water, then with brine, dried over anhydrous Na₂SO₄, filtered, and concentrated in vacuo to give the crude product 61. The crude compound 61 was used directly in the next step without further purification.

Compound 62: To a mixture of crude compound 61 (˜40 mmol) in methanol (500 mL) was added p-TsOH.H₂O (1.52 g, 8 mmol) at RT. The resulting mixture was stirred at RT overnight then was concentrated in vacuo to remove the bulk of the methanol. The residue was diluted with EtOAc, and washed with saturated NaHCO₃ solution. The aqueous phase was extracted with EtOAc, and the combined organics were washed with brine, dried over anhydrous Na₂SO₄, filtered, and concentrated in vacuo to provide the desired compound 62. The crude compound 62 was used directly in the next step without further purification.

Compound 63: To a solution of compound 62 (˜40 mmol) in DMF (200 mL) was added SOBr₂ (6.2 mL, 80 mmol) at 0° C., and the resulting mixture was stirred at 0° C. for 2 h. The mixture was then diluted with water and extracted with EtOAc. The combined organics were washed with brine, dried over anhydrous Na₂SO₄, filtered, and concentrated in vacuo. The residue was subjected to flash chromatography (silica gel, eluted by 20% to 50% EtOAc/heptane in gradient) to afford compound 63 (5.5 g, 36% overall yield for 3 steps) as a yellow solid.

Compound 64: A mixture of compound 63 (5.4 g, 14.17 mmol) and NaI (3.19 g, 21.26 mmol) in acetonitrile (300 mL) was heated under reflux conditions for 0.5 h, and then cooled to RT. To this mixture was added 1-(2,3-dichlorophenyl)piperazine hydrochloride (50; 3.73 g, 15.59 mmol) and Et₃N (2.2 mL, 15.59 mmol) at RT, and the resulting mixture was heated under reflux conditions for 3 h. The mixture was allowed to cool to RT and the precipitate was removed by filtration. The remaining filtrate was concentrated in vacuo to give the crude product 64 (12.5 g) as a light-yellow solid.

Compound 103: A solution of compound 64 (11.4 g) and AIBN (2.65 g, 16.102 mmol) in anhydrous toluene (500 mL) was infused with nitrogen for 10 min followed by the addition of n-Bu₃SnH (8.7 mL, 32.203 mmol). The resulting mixture was heated under reflux conditions for 4 h then was quenched with water. The resulting layers were separated and the aqueous layer was extracted with EtOAc. The combined organics were washed with brine, dried over anhydrous Na₂SO₄, filtered, and concentrated in vacuo. The residue was subjected to flash chromatography (Analogix machine, eluted by DCM to 5% MeOH/DCM in gradient) to provide the partially pure, desired compound. This material was further purified by flash chromatography (silica gel with dry-loading, eluted by EtOAc to 5% MeOH/EtOAc in gradient) to afford the pure compound 103 (1.7 g) as a white solid.

Compound 103 HCl salt: A mixture of compound 103 (1.7 g) in 4M HCl/dioxane (100 mL) was stirred at RT for 2 h. Solvent was removed in vacuo and the remaining residue was dried under vacuum at 50° C. to afford the target compound 103 HCl salt (1.9 g) as an off-white solid. ¹H-NMR (300 MHz, DMSO-d₆): δ 1.76-1.87 (m, 4H), 2.40 (s, 2H), 3.14-3.17 (m, 4H), 3.42-3.61 (m, 4H), 3.94 (t, J=5.8 Hz, 2H), 6.44 (d, J=2.0 Hz, 1H), 6.50 (dd, J₁=8.2 Hz, J_{2=2.3} Hz, 1H), 7.06 (d, J=8.2 Hz, 1H), 7.22 (dd, J₁=6.8 Hz, J₂=2.6 Hz, 1H), 7.36-7.38 (m, 2H), 10.04 (s, 1H), 10.50 (bs, 1H). ¹³C-NMR (75 MHz, DMSO-d₆): δ 20.68, 26.52, 31.24, 48.44, 51.74, 67.02, 67.34, 102.39, 108.12, 116.24, 120.52, 126.01, 126.72, 129.11, 129.36, 133.42, 139.94, 150.18, 158.37, 171.02. HPLC (method: 20 mm C18-RP column; mobile phase: 2-95% ACN+0.1% formic acid in 3.3 min with 1.7 min hold at 95% ACN, Wavelength: 254 nm): retention time: 2.78 min. MS (M+H⁺): 452.1. Elemental Analysis (C₂₃H₂₃D₄Cl₂N₃O₂.HCl.1.9H₂O): Calculated: C=52.81, H=6.13, N=8.03. Found: C=53.06, H=6.10, N=7.73.

### Synthesis of 7-[4-[4-(2,3-Dichlorophenyl)-1-piperazinylo]butoxy]-3,4-d₄-3,4-dihydro-2(1H)-quinolinone hydrochloride salt (Compound 104 HCl Salt)

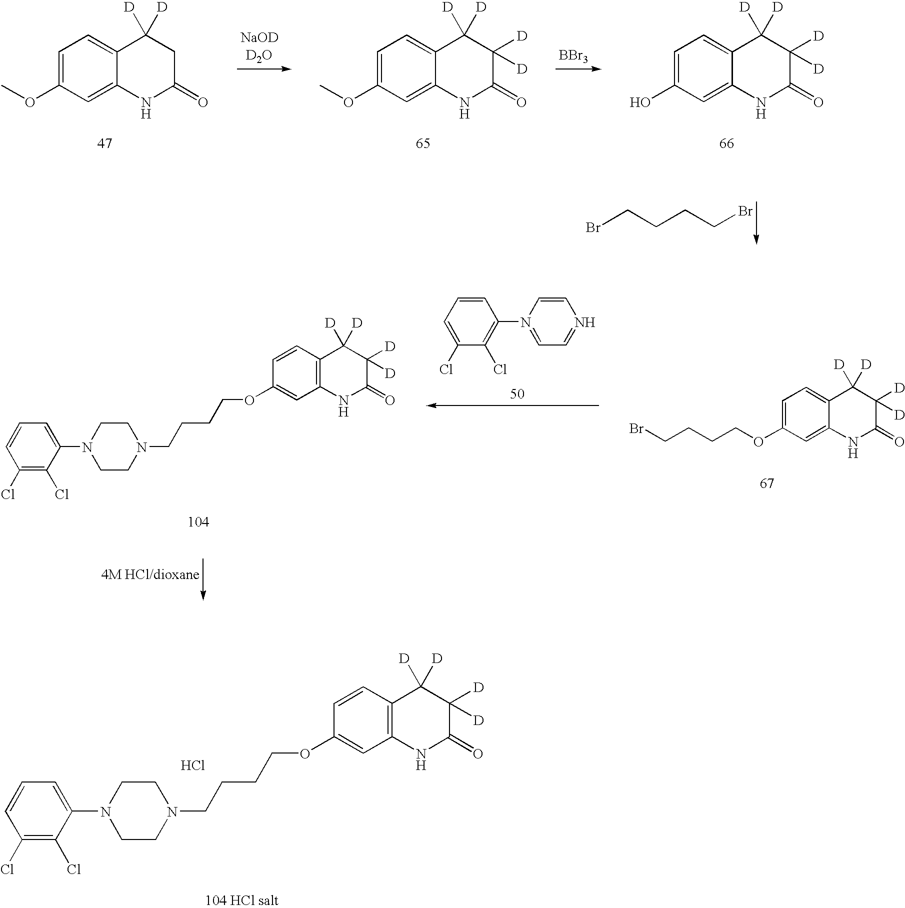

Compound 65: A mixture of compound 47 (9.0 g, 50 mmol) and NaOD (40% solution in D₂O, 25 mL, 250 mmol) in D₂O (100 mL) was heated at 100° C. in a sealed tube for 2 days. The mixture was made acidic with 35% DCl solution in D₂O under the cooling of an ice-bath, then was extracted with EtOAc. The combined organics were washed with brine, dried over anhydrous Na₂SO₄, filtered, and concentrated in vacuo to afford the desired compound 65 (9.5 g).

Compound 66: To a solution of compound 65 (24.3 g, 0.1341 mol) in DCM (750 mL) was added BBr₃ (1.0M solution in DCM, 670 mL, 0.670 mol) at −70° C. The resulting mixture was allowed to warm to RT and was stirred at RT for 0.5 h. The reaction was quenched by the addition of water, and the resulting solid was filtered, washed with water, and dried under vacuum at 60° C. to afford the desired compound 66 (20.5 g, 91% yield) as a grey solid.

Compound 67: A mixture of compound 66 (5.02 g, 30 mmol), KOH (2.24 g, 40 mmol), and 1,4-dibromobutane (10.8 mL, 90 mmol) in isopropanol (150 mL) was heated under reflux conditions for 3 h. The resulting solid was removed by filtration, and the filtrate was concentrated in vacuo. The residue was triturated with heptane to give the desired compound 67 (6.34 g, 70% yield) as a white solid.

Compound 104: A mixture of compound 67 (1.73 g, 5.725 mmol) and NaI (1.29 g, 8.587 mmol) in acetonitrile (100 mL) was heated under reflux conditions for 0.5 h, and then cooled to RT. To this mixture was added 1-(2,3-dichlorophenyl)piperazine hydrochloride (1.44 g, 6.011 mmol) and Et₃N (0.92 mL, 6.612 mmol) at RT, and the resulting mixture was heated under reflux conditions for 3 h. The mixture was allowed to cool to RT and the resulting solid was removed by filtration. The filtrate was concentrated in vacuo and the remaining residue was subjected to flash chromatography (silica gel with dry-loading, eluted by EtOAc to 5% MeOH/EtOAc in gradient) to afford compound 104 (1.8 g, 70% yield) as a white solid. ¹H-NMR (300 MHz, CDCl3): δ 1.72-1.83 (m, 4H), 2.48-2.50 (m, 2H), 2.66 (bs, 4H), 3.08 (bs, 4H), 3.97 (bs, 2H), 6.30 (s, 1H), 6.53 (d, J=7.9 Hz, 1H), 6.95-6.97 (m, 1H), 7.05 (d, J=7.9 Hz, 1H), 7.15-7.17 (m, 2H), 7.26 (d, J=1.5 Hz, 1H), 7.72 (bs, 1H). ¹³C-NMR (75 MHz, CDCl3): δ 23.62, 27.47, 51.53, 53.51, 58.38, 68.14, 77.42, 102.33, 108.86, 115.85, 118.80, 124.79, 127.65, 127.75, 128.95, 134.27, 138.33, 151.51, 158.91, 171.66. HPLC (method: 20 mm C18-RP column; mobile phase: 2-95% ACN+0.1% formic acid in 3.3 min with 2 min hold at 95% ACN, Wavelength: 254 nm): retention time: 2.78 min. MS (M+H⁺): 452.1. Elemental Analysis (C₂₃H₂₃D₄Cl₂N₃O₂): Calculated: C=61.06, H=6.03, N=9.29. Found: C=61.06, H=5.93, N=9.09.

Compound 104 HCl salt: A mixture of compound 104 (3.8 g) in 4M HCl/dioxane (100 mL) was stirred at RT for 2 h. Solvent was removed in vacuo and the remaining residue was dried under vacuum at 50° C. to afford the target compound 104 HCl salt (4.0 g) as an off-white solid. ¹H-NMR (300 MHz, DMSO-d₆): δ 1.73-1.91 (m, 4H), 3.11-3.28 (m, 4H), 3.42 (d, J=10.8 Hz, 1H), 3.59 (d, J=10.5 Hz, 1H), 3.94 (t, J=5.8 Hz, 2H), 6.46-6.50 (m, 2H), 6.52 (d, J=8.3 Hz, 1H), 7.06 (d, J=8.2 Hz, 1H), 7.21 (dd, J₁=6.8 Hz, J₂=2.7 Hz, 1H), 7.33-7.40 (m, 2H), 10.05 (s, 1H), 11.14 (bs, 1H). ¹³C-NMR (75 MHz, DMSO-d₆): δ 20.80, 26.62, 48.34, 51.71, 55.84, 67.40, 102.42, 108.16, 116.23, 120.48, 125.97, 126.70, 129.12, 129.37, 133.42, 139.94, 150.24, 158.38, 171.04. HPLC (method: 20 mm C18-RP column; mobile phase: 2-95% ACN+0.1% formic acid in 3.3 min with 1.7 min hold at 95% ACN, Wavelength: 254 nm): retention time: 2.81 min. MS (M+H⁺): 452.1. Elemental Analysis (C₂₃H₂₃D₄Cl₂N₃O₂.1HCl.1H₂O): Calculated: C=54.50, H=5.97, N=8.29. Found: C=54.39, H=5.94, N=8.13.

### Synthesis of 7-[4-[4-(2,3-Dichloro-phenyl)-1-piperazinylo]-4-d₂-butoxy]-3,4-d₄-3,4-dihydro-2(1H)-quinolinone hydrochloride salt (Compound 105 HCl Salt)

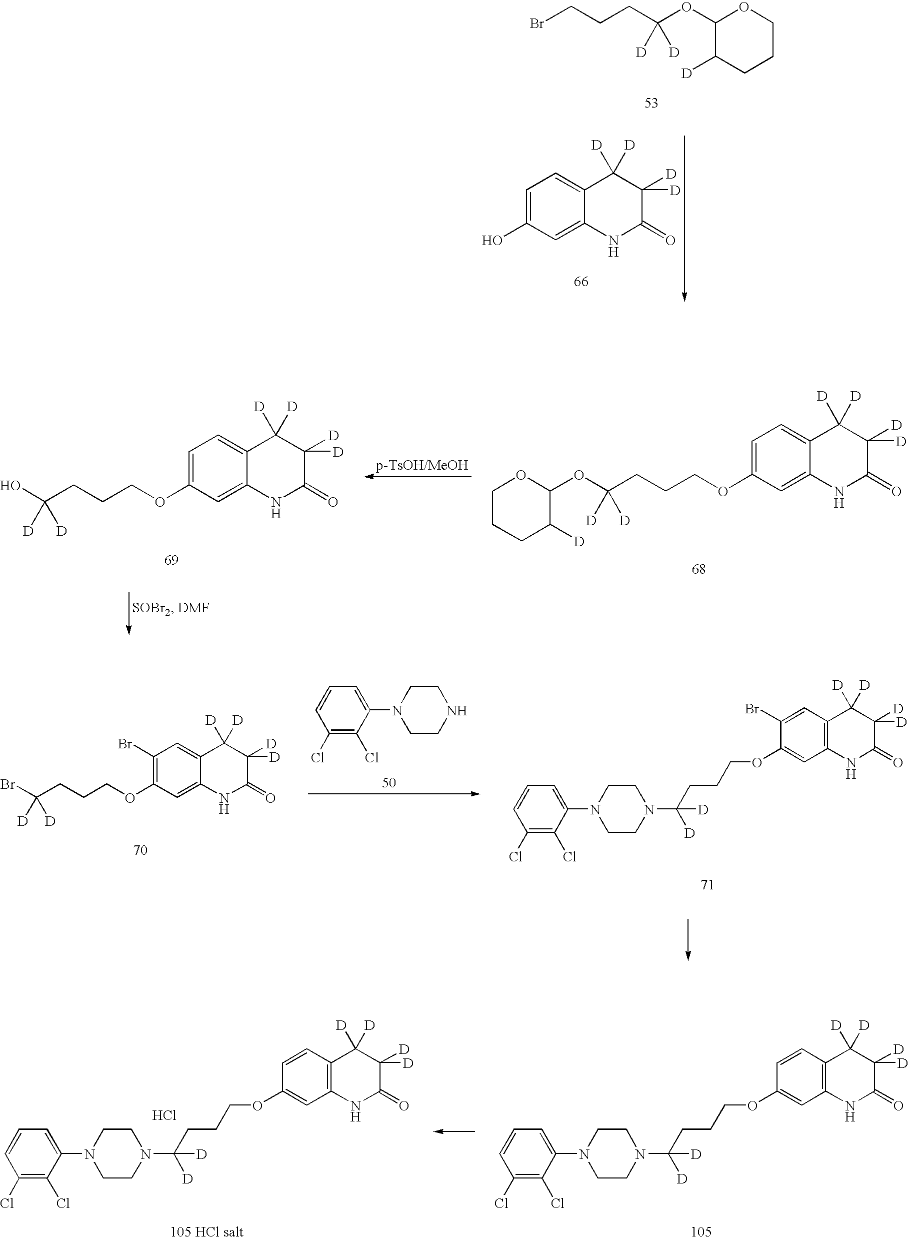

Compound 68: To a mixture of compound 66 (6.68 g, 40 mmol) in DMF (150 mL) was added NaOH (1.76 g, 44 mmol) and bromide 53 (28.8 g, 120 mmol) at RT. The resulting mixture was stirred at 50-60° C. for one day then was concentrated in vacuo to remove most of the DMF. The residue was diluted with EtOAc, washed with water, then with brine, dried over anhydrous Na₂SO₄, filtered, and concentrated in vacuo to give the crude product 68. The crude compound 68 was used directly in the next step without further purification.

Compound 69: To a mixture of crude compound 68 (˜40 mmol) in methanol (500 mL) was added p-TsOH.H₂O (1.52 g, 8 mmol) at RT. The resulting mixture was stirred at RT overnight then was concentrated in vacuo to remove most of the methanol. The residue was diluted with EtOAc and washed with saturated NaHCO₃ solution. The aqueous phase was extracted with EtOAc, and the combined organics were washed with brine, dried over anhydrous Na₂SO₄, filtered, and concentrated in vacuo to provide the desired compound 69. The crude compound 69 was used directly in the next step without further purification.

Compound 70: To a mixture of compound 69 (40 mmol) in DMF (200 mL) was added SOBr₂ (6.2 mL, 80 mmol) at −10° C. The resulting mixture was stirred at −10° C. for 1 h, then was diluted with water and extracted with EtOAc. The combined organics were washed with brine, dried over anhydrous Na₂SO₄, filtered, and concentrated in vacuo. The residue was subjected to flash chromatography (silica gel, eluted by 20% to 40% to 50% EtOAc/heptane in gradient) to afford compound 70 (8.8 g, 57% overall yield for 3 steps) as a yellow solid.

Compound 71: A mixture of compound 70 (8.8 g, 22.98 mmol) and NaI (5.17 g, 34.47 mmol) in acetonitrile (500 mL) was heated under reflux conditions for 0.5 h, and then cooled to RT. To this mixture was added 1-(2,3-dichlorophenyl)piperazine hydrochloride (50; 6.05 g, 25.30 mmol) and Et₃N (3.53 mL, 25.30 mmol) at RT, and the resulting mixture was heated under reflux conditions for 3 h. The mixture was allowed to cool to RT and the remaining solid was removed by filtration. The filtrate was concentrated in vacuo to give the crude product 71 (17.7 g) as a light-yellow solid.

Compound 105: A solution of compound 71 (17.7 g, ˜23 mmol) and AIBN (3.8 g, 23 mmol) in anhydrous toluene (600 mL) was infused with nitrogen for 10 min followed by the addition of n-Bu₃SnH (12.4 mL, 46 mmol). The resulting mixture was heated under reflux conditions for 4 h then was quenched by the addition of water. The aqueous layer was extracted with EtOAc and the combined organics were washed with brine, dried over anhydrous Na₂SO₄, filtered, and concentrated in vacuo. The residue was subjected to flash chromatography (Analogix machine, eluted by DCM to 5% MeOH/DCM in gradient) to provide the partially pure, desired compound. This material was further purified by flash chromatography (silica gel with dry-loading, eluted by EtOAc to 5% MeOH/EtOAc in gradient) to afford the pure compound 105 (3.6 g) as a white solid.

Compound 105 HCl salt: A mixture of compound 105 (3.6 g) in 4M HCl/dioxane (100 mL) was stirred at rt for 2 h. The solvent was removed in vacuo and the residue was dried under vacuum at 50° C. to afford the target compound 105 HCl salt (3.8 g) as an off-white solid. ¹H-NMR (300 MHz, DMSO-d₆): δ 1.75-1.91 (m, 4H), 3.11-3.26 (m, 4H), 3.43 (d, J=10.4 Hz, 1H), 3.58 (d, J=9.4 Hz, 1H), 3.94 (t, J=5.6 Hz, 2H), 6.46-6.52 (m, 2H), 7.06 (d, J=8.1 Hz, 1H), 7.21 (dd, J₁=6.9 Hz, J₂=2.5 Hz, 1H), 7.33-7.40 (m, 2H), 10.04 (s, 1H), 10.97 (bs, 1H). ¹³C-NMR (75 MHz, DMSO-d₆): δ 19.83, 25.78, 47.58, 50.87, 66.24, 66.60, 101.62, 107.35, 115.44, 119.70, 125.20, 125.92, 128.33, 128.58, 132.63, 139.16, 149.44, 157.59, 170.26. HPLC (method: 20 mm C18-RP column; mobile phase: 2-95% ACN+0.1% formic acid in 3.3 min with 1.7 min hold at 95% ACN, Wavelength: 254 nm): retention time: 2.77 min. MS (M+H⁺): 454.2. Elemental Analysis (C₂₃H₂₁D₆Cl₂N₃O₂.1HCl.2H₂O): Calculated: C=52.43, H=6.12, N=7.98. Found: C=52.62, H=5.85, N=7.64.

### Synthesis of 7-[4-4-(2,3-dichlorophenyl)-1-piperazinylo]butoxy]-2(1H)-quinolinone (dehydroaripiprazole)

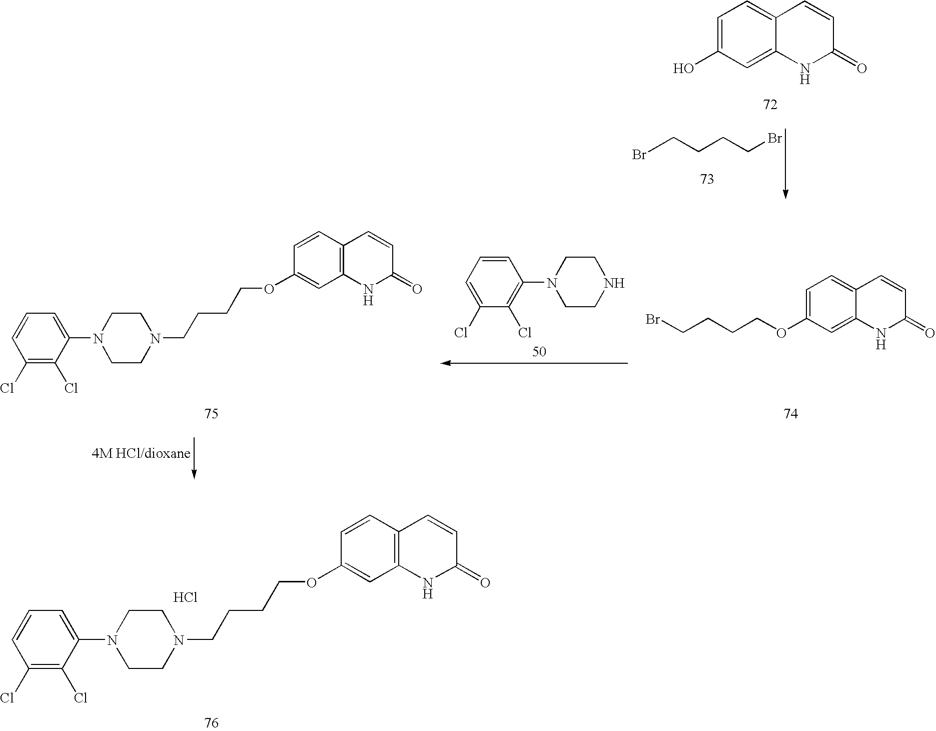

Compound 74: A mixture of 7-hydroxy-2(1H)-quinolinone (72; 6.44 g, 40 mmol), KOH (2.80 g, 50 mmol), and 1,4-dibromobutane (73; 14.4 mL, 120 mmol) in isopropanol (180 mL) was heated under reflux conditions for 6 h. The solid was removed by filtration, and the filtrate was concentrated in vacuo. The residue was triturated with heptane to give the crude compound 74 (9.70 g, ˜70% purity by LCMS) as a reddish-brown solid.

Compound 75 (dehydroaripiprazole): A mixture of compound 74 (9.70 g, ˜30 mmol) and NaI (6.75 g, 45 mmol) in acetonitrile (500 mL) was heated under reflux conditions for 0.5 h, and then cooled to RT. To this mixture was added 1-(2,3-dichlorophenyl)piperazine hydrochloride 50 (7.89 g, 33 mmol) and Et₃N (4.6 mL, 33 mmol) at RT. The resulting mixture was heated under reflux conditions for 3 h then was allowed to cool to RT. The resulting solid was removed by filtration and the filtrate was concentrated in vacuo. The residue was subjected to flash chromatography (silica gel with dry-loading, eluted by EtOAc to 5% MeOH/EtOAc in gradient) to afford the desired product. This material was further purified by re-crystallization to afford pure compound 75 (1.2 g) as an off-white solid. (GB-12-97)

Compound 75 HCl salt: A mixture of compound 75 (1.2 g) in 4M HCl/dioxane (100 mL) was stirred at RT for 2 h. The solvent was removed under reduced pressure and the residue was dried under vacuum at 50° C. to afford the HCl salt of compound 75 (1.2 g) as a light-yellow solid. ¹H-NMR (300 MHz, DMSO-d₆): δ 1.82-1.92 (m, 4H), 3.12-3.26 (m, 6H), 3.45 (d, J=9.4 Hz, 2H), 3.62 (d, J=8.5 Hz, 2H), 4.07 (t, J=5.5 Hz, 2H), 6.32 (d, J=9.4 Hz, 1H), 6.82 (dd, J₁=4.5 Hz, J₂=2.4 Hz, 2H), 7.22 (dd, J₁=6.7 Hz, J_{2=2.6} Hz, 1H), 7.34-7.41 (m, 2H), 7.58 (d, J=9.6 Hz, 1H), 7.83 (d, J=9.6 Hz, 1H), 10.52 (bs, 1H), 11.69 (bs, 1H). ¹³C-NMR (75 MHz, DMSO-d₆): δ 20.84, 26.46, 48.44, 51.81, 55.84, 67.65, 99.33, 111.47, 114.09, 119.22, 120.53, 126.01, 126.72, 129.37, 129.99, 133.42, 140.76, 141.30, 150.18, 160.94, 162.94. HPLC (method: 20 mm C18-RP column; mobile phase: 2-95% ACN+0.1% formic acid in 3.3 min with 1.7 min hold at 95% ACN, Wavelength: 254 nm): retention time: 2.74 min. MS (M+H⁺): 446.1.

Evaluation of Metabolic Stability. Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., Absorption Systems L.P. (Exton, Pa.)). The incubation mixtures are prepared as follows:

Reaction Mixture Composition

| | | |
|---|---|---|
| Liver Microsomes | 1.0 | mg/mL |
| NADPH | 1 | mM |
| Potassium Phosphate, pH 7.4 | 100 | mM |
| Magnesium Chloride | 10 | mM |
| Test Compound | 1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (not the test compound). The reaction is initiated by the addition of cofactors (not into the negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as sunitinib, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I or II in 100 mM potassium phosphate buffer (pH 7.4) was incubated at 37° C. in triplicate. Positive controls contain 1 μM or aripiprazole or dehydroaripiprazole instead of a compound of formula I or II, respectively. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

We claim:

1. A compound of formula I: 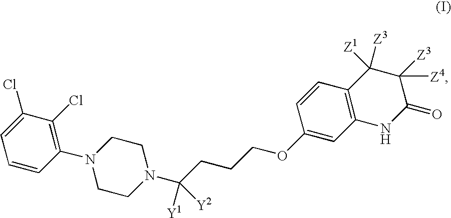
or a pharmaceutically acceptable salt, solvate, or hydrate thereof, wherein:
each Y is independently selected from hydrogen, deuterium, and fluorine; and
each Z is independently selected from hydrogen, deuterium, and fluorine, wherein:
at least one Y or Z is deuterium.

2. The compound of claim 1, wherein each of Y¹ and Y² is the same.

3. The compound of claim 2, wherein Y¹ and Y² are simultaneously deuterium.

4. The compound of any one of claims 1 to 3, wherein each of Z¹ and Z² is the same.

5. The compound of claim 4, wherein Z¹ and Z² are simultaneously deuterium.

6. The compound of claim 4, wherein Z¹ and Z² are simultaneously fluorine.

7. The compound of any one of claims 1 to 6, wherein each of Z³ and Z⁴ is the same.

8. The compound of claim 8, wherein Z³ and Z⁴ are simultaneously deuterium.

9. The compound of claim 1, wherein at least two of Y¹, Y², Z¹, Z², Z³, and Z⁴ are deuterium.

10. The compound of claim 9, wherein at least three of Y¹, Y², Z¹, Z², Z³, and Z⁴ are deuterium.

11. The compound of claim 9 or 10, wherein at least one of Y¹, Y², Z¹, Z², Z³, and Z⁴ is a fluorine atom.

12. The compound of claim 11, wherein at least two of Y¹, Y², Z¹, Z², Z³, and Z⁴ are fluorine atoms.

13. A compound of formula II: 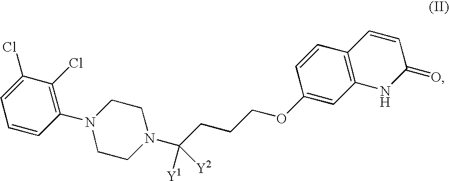
or a pharmaceutically acceptable salt, solvate, or hydrate thereof, wherein:
Y³ is deuterium; and
Y⁴ is selected from hydrogen, deuterium, or fluorine.

14. The compound of claim 13, wherein Y⁴ is deuterium.

15. The compound of claim 13, wherein Y⁴ is fluorine.

16. A compound of this invention selected from the group consisting of: 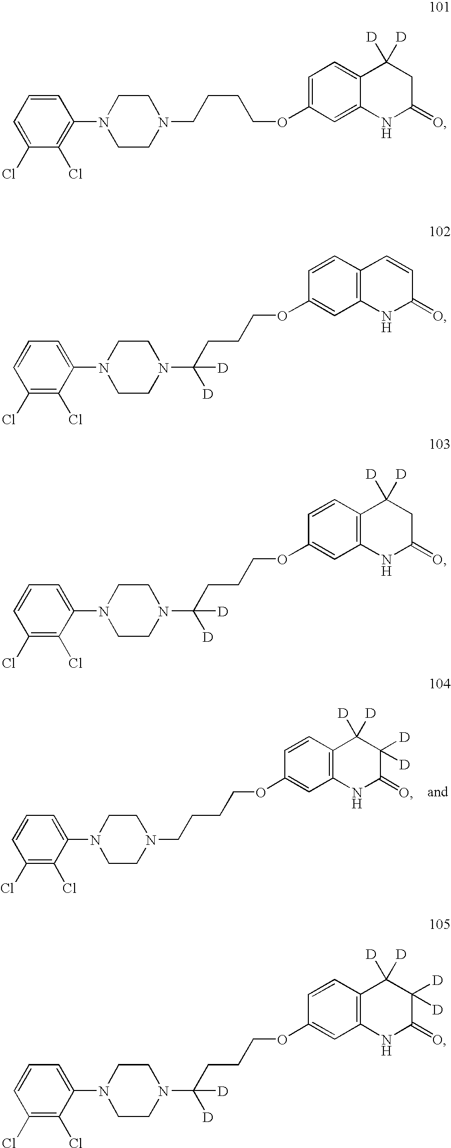
or an HCl salt thereof.

17. The compound of any one of claims 1 to 16, wherein each atom not designated as deuterium is present at its naturally abundant isotopic state.

18. A composition comprising:
a. an effective amount of a compound of any one of claims 1 to 17; and
b. an acceptable carrier.

19. The composition of claim 18, wherein said composition is formulated for pharmaceutical use; and the carrier is a pharmaceutically acceptable carrier.

20. The composition of claim 19, wherein the composition is formulated for oral administration.

21. The composition of claim 20, wherein the composition is in the form of a pill, capsule or tablet.

22. The composition of claim 21 in dosage unit form, comprising from 0.1 to 250 mg of said compound.

23. The composition of claim 22, comprising from 2 to 50 mg of said compound.

24. The composition of claim 19 further comprising an effective amount of a second therapeutic agent, wherein said second therapeutic agent is useful for treating or preventing a disease or a condition selected from schizophrenia, depression, bipolar depression, depressive disorder, refractive bipolar disorder, autism, alcoholism, cocaine dependency, attention deficit hyperactivity disorder, mood disorders, post traumatic stress disorder, premenstrual dysphoric disorder, nausea, psychotic disorder, tardive dyskinesia, epilepsy, compulsivity, impulsivity, cognition enhancement, weight management, sexual disorders including Hypoactive Sexual Desire Disorder, loss of sexual desire, lack of sexual desire, decreased sexual desire, inhibited sexual desire, loss of libido, libido disturbance, and frigidity.

25. The composition according to claim 24, wherein said second therapeutic agent is selected from an NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, an M1/M4 receptor agonist; an opioid antagonist; D-cycloserine; Lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing additional therapeutic agents; and combinations of the foregoing therapeutic agents.

26. A substantially isolated isomer of a compound of any one of claims 1 to 17.

27. An article of manufacture comprising separate dosage forms of:
a. a compound of any one of claims 1 to 17; and
b. a second therapeutic agent,
wherein both dosage forms are in a single container.

28. A method for treating a patient suffering from or susceptible to a disorder beneficially treated by an atypical antipsychotic agent, comprising the step of administering to the patient in need thereof a composition according to claim 19.

29. The method according to claim 28, wherein the disorder is selected from schizophrenia, mania or bipolar disorder.

30. The method according to claim 28, wherein the disorder is selected from major depressive disorder, ADHD, autism, conduct disorder, anxiety disorder, social anxiety disorder, substance abuse, prodromal psychosis, Tourette's disorder, Asperger's disorder, pervasive developmental disorder, or alcoholism.

31. The method according to any one of claims 28 to 30, comprising the additional step of coadministering to the patient in need thereof a second therapeutic agent selected from an NK3 receptor antagonist; a Gly Transporter Type I inhibitor; memantine; an AMPA receptor potentiator; a GABA modulator, anticonvulsant, or benzodiazepine; an antidepressant; a nicotinic receptor agonist or antagonist; a serotonin reuptake inhibitor; sabcomeline, an M1/M4 receptor agonist; an opioid antagonist; D-cycloserine; Lamotrigine; methylphenidate; divalproex; clozapine; H1-receptor; an adenosine A2a receptor antagonist; COX-2 inhibitor; an azabicyclo compound for treating CNS disorders; flibanserin; lithium; a pharmaceutically acceptable salt of any of the foregoing second therapeutic agents; or combinations of the foregoing second therapeutic agents.

32. The method of claim 31, wherein:
a. the patient suffering from or susceptible to schizophrenia or bipolar disorder; and the second therapeutic agent is selected from clozapine, depakote ER, or lamotrigine;
b. the patient suffering from or susceptible to ADHD; and the second therapeutic agent is methylphenidate;
c. the patient suffering from or susceptible to autism; and the second therapeutic agent is D-cycloserine; or
d. the patient suffering from or susceptible to alcoholism; and the second therapeutic agent is an opioid antagonist.

33. A pharmaceutical composition for use in the treatment of a condition selected from schizophrenia, bipolar disorder, bipolar mania, autism, alcoholism, agitation, attention deficit/hyperactivity disorder, anxiety, behavioral disorder, dementia, Alzheimer's dementia, Asperger's disorder, conduct disorder, depression, drug dependency, insulin resistance, mania, obsessive-compulsive disorder, Parkinson's disease, psychosis associated with dementia, drug-induced psychosis, pervasive developmental disorder, prodromal schizophrenia, prodromal psychoses, schizoaffective disorder, social anxiety, tic, and Tourette's disorder, said composition comprising a compound of any one of claims 1 to 17; and a pharmaceutically acceptable carrier.

34. The composition according to claim 33, wherein the use is the treatment of schizophrenia, mania or bipolar disorder.

35. The composition according to claim 33, additionally comprising a second therapeutic agent selected from clozapine, depakote ER, and lamotrigine.
